(19) United States
(12) Patent Application Publication
(10) Pub. No.: US 2026/0043808 A1
(43) Pub. Date: Feb. 12, 2026

(54) BIOSENSORS COMPRISING AMPHIPHILIC POLYMERS AND USE THEREOF

(71) Applicant: Sartorius BioAnalytical Instruments, Inc., Bohemia, NY (US)

(72) Inventor: Dan Su, Fremont, CA (US)

(73) Assignee: Santorius BioAnalytical Instruments, inc., Bohemia, NY (US)

(21) Appl. No.: 19/367,118

(22) Filed: Oct. 23, 2025

Related U.S. Application Data
(63) Continuation of application No. PCT/US2024/025188, filed on Apr. 18, 2024.
(60) Provisional application No. 63/463,091, filed on May 1, 2023.

(51) Int. Cl. G01N 33/68; G01N 21/64; G01N 33/543

(52) U.S. Cl. CPC G01N 33/6845; G01N 21/6428; G01N 33/543; G01N 33/6857; G01N 2021/6439

(57) ABSTRACT

Biosensors for detecting and analyzing protein aggregates in a sample are provided. A biosensor may be coated with an amphiphilic polymer having one or more hydrophobic groups for binding to protein aggregates, based on hydrophobic effects. An outer surface of a protein aggregate may be generally non-polar and hydrophobic and, thus, drawn to and captured by hydrophobic groups of the amphiphilic polymer. The biosensor may detect the binding of an analyte directly, without use of a detectable label attached to the analyte. In certain embodiments, the biosensor may detect the binding of the analyte based on indirect detection of a fluorescent label that binds to the analyte after capture by the biosensor.

### CROSS-REFERENCE TO RELATED APPLICATIONS

This application is a bypass continuation of WIPO Application Serial No. PCT/US2024/025188, filed Apr. 18, 202, which claims the benefit of, and priority to, U.S. Patent Application Ser. No. 63/463,091, filed May 1, 2023. The entire contents of each of the above applications are hereby incorporated by reference.

### BACKGROUND

The present disclosure relates to biosensors and, more specifically, to biosensors having amphiphilic polymers for detection and/or quantitation of compounds of interest and related methods.

Critical quality attributes (CQAs), such as size, charge, aggregation, glycosylation patterns, and process-related impurities, are vital contributors for drug discovery, development, and manufacturing. Biomolecules, such as proteins and nucleic acids, may often partially occur in the form of aggregates, which is often undesired. Traditional techniques used to measure CQAs, such as aggregation, are time and labor intensive.

Label-free biosensor systems can monitor interactions between biomolecules in real time based on optical biosensors. However, existing biosensors are mainly based on limited biochemistries, such as antibody-antigen or receptor-ligand interactions. For example, typical current analyte-anti-analyte pairs include antigen-antibody pairs or ligand-receptor pairs, where the analyte can be either member of the pair, and the biosensor is coated with the anti-analyte that is the opposite member of the pair. The analyte, such as an antigen, an antibody, a ligand, or a receptor can be detected based on its binding to the specific anti-analyte of the biosensor.

In view of the foregoing, there is an unmet demand for effective biosensors that operate via expanded classes of coating chemistries, which encompass molecular or biological interactions other than antigen-antibody or ligand-receptor interactions for sensing and quantitating a variety of compounds and characteristics of such compounds.

### SUMMARY

The disclosure herein provides embodiments of biosensors for detecting the presence or amount of protein aggregates in a sample. A biosensor may be coated with a layer of an amphiphilic polymer, in which one or more hydrophobic groups of the amphiphilic polymer is capable of binding to protein aggregates based on hydrophobic effects. For example, an outer surface of a protein aggregate may be generally non-polar or hydrophobic and, thus, drawn to and captured by hydrophobic groups of the amphiphilic polymer. In some cases, the biosensor may detect the binding of an analyte, such as protein aggregates, directly, without use of a detectable label attached to the analyte. In certain embodiments, the biosensor may detect the binding of the analyte based on indirect detection via a fluorescent label that binds to the analyte after capture by the biosensor. The present disclosure also provides apparatuses coupled to the biosensor for quantitating the signals generated by binding of the biosensor to the protein aggregate, as well as methods for detecting presence or amount of a protein aggregate in a sample using the biosensor.

In certain aspects of the present disclosure, a biosensor for detecting a presence or amount of protein aggregates in a sample is provided. The biosensor includes a core component having a distal surface and a plurality of amphiphilic polymers. Each amphiphilic polymer of the plurality of amphiphilic polymers includes a linker having a first end and a second end, and a hydrophilic moiety coupled between the first end of the linker and the distal surface of the core component. Each amphiphilic polymer of the plurality of amphiphilic polymers also includes a hydrophobic portion coupled to the second end of the linker to facilitate capture of the protein aggregates in the sample via hydrophobic interactions and to facilitate generating of signals indicative of an amount of the protein aggregates captured by the biosensor.

In some embodiments, the hydrophobic portion of each amphiphilic polymer facilitates the capture of the protein aggregates via production of a hydrophobic region for sequestering non-polar components therein. In embodiments, a fluorescent dye is bound to the captured protein aggregates of each amphiphilic polymer.

In certain embodiments, the linker includes a single linker arm. In other embodiments, the linker includes multiple linker arms that are each coupled to a respective hydrophobic portion of a plurality of hydrophobic portions.

In certain embodiments, the linker includes polyethylene glycol (PEG).

In some embodiments, the hydrophobic portion includes a phospholipid or an amine protecting group. For example, the hydrophobic portion includes 1,2-distearoyl-sn-glycero-3-phosphorylethanolamine (DSPE). As another example, the hydrophobic portion includes fluorenylmethoxycarbonyl (FMOC). The distal surface of the core component of certain embodiments includes aminopropylsilane (APS). In embodiments, the protein aggregates include aggregates of immunoglobulin G (IgG).

In certain aspects of the present disclosure, an apparatus for quantitating an aggregated protein in a sample is provided. The apparatus includes a biosensor and a signal analyzer. The biosensor includes a core component and a plurality of amphiphilic polymers. Each amphiphilic polymer of the plurality of amphiphilic polymers includes a hydrophilic moiety coupled to a distal surface of the core component and a linker coupled to the hydrophilic moiety. Each amphiphilic polymer includes at least one hydrophobic portion coupled to the linker to facilitate capture of the aggregated protein in the sample via hydrophobic interactions. The aggregated protein may be labeled with a fluorescent dye to facilitate generating of signals indicative of quantity of the aggregated protein captured by the biosensor. The signal analyzer is coupled to the biosensor and is capable of transforming and quantifying the signals generated by labeling of the aggregated protein captured by the biosensor.

In some embodiments, the signal analyzer quantifies the signals generated by labeling of the aggregated protein based on a predetermined calibration curve describing a relationship between aggregated protein concentrations and interaction parameters, such as binding rate, initial slope, and response at equilibrium.

In embodiments, the amphiphilic polymer component is immobilized on the distal surface of the core component, and the signal analyzer performs a solid-phase assay. In certain embodiments, the hydrophilic moiety is covalently bonded to the distal surface of the core component.

In some embodiments, the at least one hydrophobic portion coupled to the linker includes two or more hydrophobic portions. In certain embodiments, the at least one hydrophobic portion includes DSPE or FMOC.

In certain aspects of the present disclosure, a method for determining a presence or an amount of protein aggregates in a sample in real time and without reagent labeling is provided. The method includes contacting the sample with a biosensor. The biosensor includes a core component and a plurality of amphiphilic polymers layered on a distal surface of the core component. Each amphiphilic polymer of the plurality of amphiphilic polymers includes a hydrophilic moiety coupled to the core component, a linker coupled to the hydrophilic moiety, and a hydrophobic portion coupled to the linker. The method also includes capturing, via hydrophobic interactions, at least a portion of the protein aggregates in the sample with the hydrophobic portion of the plurality of amphiphilic polymers. The method further includes labeling the at least a portion of the protein aggregates with a fluorescent dye. The method includes determining the presence or the amount of the protein aggregates in the sample based on fluorescence intensity associated with the fluorescent dye bound to the protein aggregates.

In certain embodiments, the method includes determining the presence or the amount of the protein aggregates by measuring the fluorescence intensity associated with the fluorescent dye and converting the fluorescence intensity to a concentration based on a calibration curve. In certain embodiments, the method includes determining the presence or the amount of the protein aggregates by measuring a binding rate, initial slope, response at equilibrium, or combination thereof based on the binding of protein aggregates to the hydrophobic portion of the plurality of amphiphilic polymers.

In some embodiments, the protein aggregates include aggregated IgG. In embodiments, the fluorescent dye includes Congo Red (CR) dye or Thioflavin T (TFT) dye. In some embodiments, the hydrophobic portion of the plurality of amphiphilic polymers includes DSPE, FMOC, or ortho-pyridyl disulfide (OPSS). In embodiments, the hydrophilic moiety of the plurality of amphiphilic polymers is covalently bonded to a binding group of the distal surface of the core component.

In certain aspects of the present disclosure, a method for preparing a biosensor for analyzing protein aggregates in a sample is provided. The method includes providing a core component having a distal surface, where the distal surface includes a plurality of binding groups. The method also includes providing a plurality of amphiphilic polymers. Each amphiphilic polymer of the plurality of amphiphilic polymers includes a hydrophilic moiety, a linker coupled to the hydrophilic moiety, and a hydrophobic portion coupled to the linker. Additionally, the method includes exposing the distal surface of the core component to the plurality of amphiphilic polymers, such that the hydrophilic moiety of the plurality of amphiphilic polymers binds to the plurality of binding groups of the distal surface and the hydrophobic portion of the plurality of amphiphilic polymers is immobilized to facilitate analysis of protein aggregates.

In some embodiments, the hydrophilic moiety of the plurality of amphiphilic polymers or the binding group of the distal surface includes an amine group. In some embodiments, the hydrophobic portion of the plurality of amphiphilic polymers includes DSPE, FMOC, or OPSS.

In some embodiments, the method includes preparing the plurality of amphiphilic polymers by binding the hydrophilic moiety to a first end of the linker and binding the hydrophobic portion to a second end of the linker. In embodiments, the method includes capturing, via hydrophobic interactions, at least a portion of protein aggregates in a sample with the hydrophobic portion of the plurality of amphiphilic polymers, labeling the at least a portion of the protein aggregates with a fluorescent dye, and determining a presence or an amount of the protein aggregates in the sample based on signals emitted from the fluorescent dye.

### BRIEF DESCRIPTION OF THE DRAWINGS

Various aspects of the present disclosure are described hereinbelow with reference to the drawings, which are incorporated in and constitute a part of this specification.

FIG. 1 schematically depicts an example process of coating a biosensor with amphiphilic polymers for capturing certain protein aggregates, in accordance with the present disclosure.

FIG. 2 schematically depicts another example process of coating a biosensor with amphiphilic polymers for capturing certain protein aggregates, in accordance with the present disclosure.

FIG. 3 depicts a detailed schematic view of a biosensor for capturing aggregated proteins via an amphiphilic polymer, in accordance with the present disclosure.

FIG. 4A depicts a schematic view of an amphiphilic polymer having a linker with two linker arms, in accordance with the present disclosure.

FIG. 4B is a schematic view of another amphiphilic polymer having a linker with four linker arms, in accordance with the present disclosure.

FIG. 5 depicts an example process of detecting the presence and/or amount of protein aggregates in a sample, in accordance with the present disclosure.

FIG. 6 depicts an example process of detecting the presence and/or amount of protein aggregates in a sample with a detectable label, in accordance with the present disclosure.

FIG. 7A is a graphical representation of binding curves illustrating a change in wavelength (nanometer (“nm”) shift) versus time for capturing various concentrations of protein aggregates via a first amphiphilic polymer, in accordance with the present disclosure.

FIG. 7B is a graphical representation of an embodiment of a calibration curve, which depicts a relationship of binding rate versus aggregate concentration from FIG. 7A, in accordance with the present disclosure.

FIG. 8A is a graphical representation of binding curves illustrating a change in wavelength (nm shift) versus time for capturing various concentration of protein aggregates via a second amphiphilic polymer, in accordance with the present disclosure.

FIG. 8B is a graphical representation of an embodiment of a calibration curve, which depicts a relationship of binding rate versus aggregate concentration from FIG. 8A, in accordance with the present disclosure.

### DETAILED DESCRIPTION

The present disclosure will now be described more fully hereinafter with reference to example embodiments thereof and with reference to the drawings. These example embodiments are described so that this disclosure will be thorough and complete, and will fully convey the scope of the disclosure to those skilled in the art. Features from one embodiment or aspect may be combined with features from any other embodiment or aspect in any appropriate combination. For example, any individual or collective features of method aspects or embodiments may be applied to apparatus, product, or component aspects or embodiments and vice versa. The disclosure may be embodied in many different forms and should not be construed as limited to the embodiments set forth herein; rather, these embodiments are provided so that this disclosure will satisfy applicable legal requirements.

A “biosensor” as used herein refers to a sensing and/or analytical device that detects the presence, characteristics, or amount of substances (analytes) using a particular molecule (such as an enzyme, an antibody, a biopolymer, or an amphiphilic polymer) or a living organism. Biosensors may employ a solid surface having immobilized anti-analyte molecules to which sample analyte molecules bind specifically and with high affinity at a defined detection zone. In this type of assay, known as a solid-phase assay, the solid surface is exposed to the sample under conditions that promote analyte binding to immobilized anti-analyte molecules. The binding event may be detected directly, e.g., by a change in the mass, reflectivity, thickness, color, or other characteristic indicative of a binding event. Where the analyte is pre-labeled, for example with a dye, a chromophore, a fluorophore, or radiolabel, the binding event is detectable by the presence and/or amount of detectable label at the detection zone. Alternatively, the analyte may be labeled after it is bound at the detection zone, for example with a secondary, fluorescent-labeled anti-analyte antibody.

The term “coat” or “load” in the context of coating or loading a structure with a polymer refers to the process of bringing the equilibrated sample (such as an amphiphilic polymer) into contact with the equilibrated solid phase (such as to assemble a biosensor). Coating or loading may be done with chromatography devices by causing the sample to pass through the device by means of an external force, such as by gravity; by pumping; or by dipping into a well plate that contains the sample.

A “binding” or “binding reaction” refers to an attractive interaction between two molecules resulting in a stable association in which the molecules are in close proximity to each other. Binding is saturable, may be reversible, and may be competed with an excess of one of the reactants. Binding reactions are characterized by complementarity of shape, polarity, electronegativity, and other binding determinants as between the participants in the specific binding reaction.

An “analyte” as used herein refers to a substance, the presence, characteristics, or amount of which is analyzed via the biosensor provided herein. An “analyte-binding” molecule or an “anti-analyte” as used herein refers to a molecule or a compound capable of participating in a specific binding reaction with an analyte molecule. Analyte-anti-analyte pairs may include antigen-antibody pairs, ligand-receptor pairs, or complementary polynucleotide pairs where the analyte may be either member of the pair and the anti-analyte is the opposite member of the pair. An anti-analyte may be immobilized or coated onto the biosensor for binding and detection of an analyte.

A “polymer” as used herein refers to a molecule or macromolecule formed from monomeric units that are covalently bonded in one or more chains. The polymer may be a naturally derived biopolymer or a synthetic polymer. “Hydrophilic” as used herein with reference to a functional group, moiety, molecule, or polymer refers to an entity that is polar and attracted by water, such that the entity is soluble in polar solvents including water. “Hydrophobic” as used herein with reference to a functional group, moiety, molecule, or polymer refers to an entity that is non-polar and repelled by water, such that the entity is insoluble in polar solvents. When added to a polar solvent, non-polar entities or materials generally aggregate or cluster together based on the hydrophobic effect.

Non-limiting examples of hydrophobic functional groups, moieties, molecules, or polymers contemplated herein comprise phospholipids including 1,2-distearoyl-sn-glycero-3-phosphorylethanolamine (DSPE), amine protecting groups including (i) fluorenylmethoxycarbonyl (FMOC) and (ii) di-tert-butyl dicarbonate (BOC), amine reactive groups including dibenzocyclooctyne-amine (DBCO), thiol-reactive groups including ortho-pyridyl disulfide (OPSS), sterols including cholesterol, polyesters including polylactic acid (PLA), nitrophenols including 4-hydroxy-3-nitrophenylacetyl-epsilon-aminocaproic acid (NPC), aromatic hydrocarbons including pyrene, and aliphatic hydrocarbons including (i) C12, (ii) C16, and (iii) C18.

“Amphiphilic” as used herein with reference to a polymer, compound, or molecule refers to a polymer having properties of both hydrophobic functional groups and hydrophilic functional groups. An amphiphilic polymer comprises at least one hydrophobic functional group, moiety, head, or polymer and at least one hydrophilic functional group, moiety, head, or polymer. For example, the amphiphilic polymer may include a hydrophobic functional group, as described above, chemically bound to a hydrophilic functional group. In some embodiments, the hydrophilic functional group is coupled to a linker, cross-linker, or spacer component for separating the hydrophobic component from a particular element, such as a surface or binding group thereon to which an amphiphilic polymer is bound. The linker may include a linear, branched, Y-shaped, or multi-arm structure or geometry. A non-limiting example of a linker includes polyethylene glycol (PEG). In some examples, the linker includes PEG methyl ether (mPEG), poly-2-oxazoline (POX), polyvinylpyrrolidone (PVP), polypropylene glycol (PG), polyacrylamide, polyaminoacid, polycarboxybetaine, and/or polysulfobetaine.

A “protein” as used herein refers to a chemical compound formed of amino acids, which are bonded together into one or more chain. A three-dimensional structure of the protein may be dependent on the sequence of amino acids, hydrogen bonds between non-sequential amino acids, and/or folding of the chains thereof to generate an overall shape. A “protein aggregate” as used herein refers to a collection of multiple proteins or fragments thereof that have at least partially unfolded and accumulated into a singular mass, such as based on interactions between unfolded regions.

An “antibody” refers to an example of a protein, such as an immunoglobulin (lg) molecule having two heavy chains and two light chains prepared by any method known in the art or later developed. Antibodies may include IgG, IgM, IgA, IgD, and IgE. Antibodies also include polyclonal antibodies such as those produced by inoculating a mammal such as a goat, mouse, rabbit, etc. with an immunogen, as well as monoclonal antibodies produced using the well-known Kohler Milstein hybridoma fusion technique. The term includes antibodies produced using genetic engineering methods such as those employing, e.g., SCID mice reconstituted with human immunoglobulin genes, as well as antibodies that have been humanized using art-known resurfacing techniques. An antibody also refers to an antibody fragment. An “antibody fragment” refers to a fragment of an antibody molecule produced by chemical cleavage or genetic engineering techniques, as well as to single chain variable fragments (SCFvs) such as those produced using combinatorial genetic libraries and phage display technologies. Antibody fragments used in accordance with the present disclosure may retain the ability to bind their cognate antigen and so include variable sequences and antigen combining sites, which are within the scope of antibodies.

As used herein, the term “proximal” refers to the portion of the device or component thereof that is closer to the user or machine using the device. The term “distal” refers to the portion of the device or component thereof that is farther from the user or machine using the device. As used herein, the singular forms “a,” “an,” “the,” and the like include plural referents unless the context clearly dictates otherwise. In addition, while reference may be made herein to quantitative measures, values, geometric relationships, or the like, unless otherwise stated, any one or more if not all of these may be absolute or approximate to account for acceptable variations that may occur, such as those due to manufacturing or engineering tolerances or the like. Reference to a value or parameter herein includes (and describes) embodiments that are directed to that value or parameter per se with in a range suitable in the context, for example ±10%. For example, description referring to “X” includes description of “X” and extends to a suitable range.

### 1. Biosensor Comprising a Layer of Amphiphilic Polymer

A biosensor of the present disclosure, for detecting presence or amount of a protein aggregate in a sample, includes a core component having a distal surface and a plurality of amphiphilic polymers. Each amphiphilic polymer of the plurality of amphiphilic polymers includes a linker having a first end and a second end, and a hydrophilic moiety coupled between the first end of the linker and a binding group of the distal surface of the core component. Each amphiphilic polymer of the plurality of amphiphilic polymers also includes a hydrophobic portion coupled to the second end of the linker. As such, capture of the protein aggregates in the sample via hydrophobic interactions and generating of signals indicative of an amount of the protein aggregates captured by the biosensor are facilitated.

In some embodiments, the hydrophobic portion of each amphiphilic polymer facilitates the capture of the protein aggregates via production of a hydrophobic region for sequestering non-polar components therein. In embodiments, a fluorescent dye is bound to the captured protein aggregates of each amphiphilic polymer. In certain embodiments, the linker includes a single linker arm. In other embodiments, the linker includes multiple linker arms that are each coupled to a respective hydrophobic portion of a plurality of hydrophobic portions. In some embodiments, the hydrophobic portion includes a phospholipid or an amine protecting group. For example, the hydrophobic portion may include DSPE. As another example, the hydrophobic portion may include FMOC. The distal surface of the core component of certain embodiments includes APS. In embodiments, the protein aggregates include aggregates of IgG.

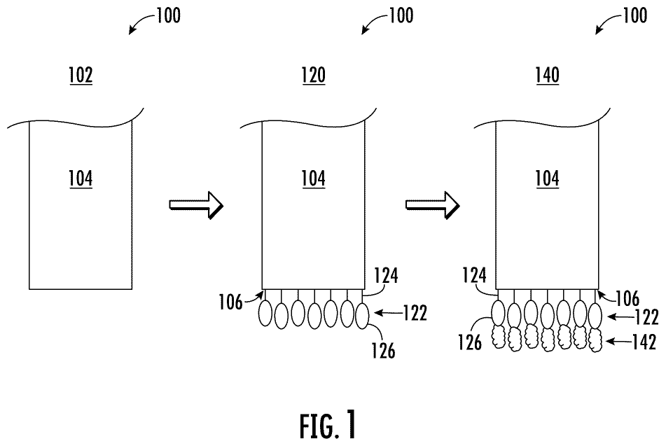

FIG. 1 schematically depicts an embodiment of a process of coating a biosensor 100 with amphiphilic polymers for capturing certain analytes, such as protein aggregates. As illustrated in a first phase 102, the biosensor 100 is provided with a core component 104 that may be formed of and/or be coated with one or more suitable polymers or coatings. The core component 104 includes a distal surface 106 containing binding groups that may be selectively exposed to samples, such as fluids within a well plate. For example, a coating may be provided onto the core component 104, such that the distal surface 106 is suitably prepared for immobilizing amphiphilic polymers thereto. In embodiments, the core component 104 and/or the distal surface 106 thereof includes aminopropylsilane (APS). In embodiments, the core component 104 and/or the distal surface 106 includes epoxypropylsilane (EPS). In embodiments, the core component 104 and/or the distal surface includes any suitable silane treated sensor. In some embodiments, the binding groups of the distal surface 106 may bind with or be coated, covered, or layered with a suitable polymer, molecule, or compound to function as an anti-analyte for facilitating subsequent analyte analysis via interaction with the coating. For example, in a second phase 120, the distal surface 106 may be coated with amphiphilic polymers 122 via a suitable immobilization process. The amphiphilic polymers 122 may be formed in a layer having a target thickness.

As recognized herein, the amphiphilic polymers 122 each include at least one hydrophilic portion 124 for coupling to the biosensor 100 and at least one hydrophobic portion 126 for binding with an analyte. In embodiments, the hydrophilic portion 124 is a functional group, moiety, or polymer of the amphiphilic polymer 122 including at least one component that is generally polar, while the hydrophobic portion 126 is a functional group, moiety, or polymer of the amphiphilic polymer 122 including at least one component that is generally non-polar. The hydrophilic portion 124 may include at least one hydrophilic moiety and a linker extending between the hydrophilic moiety and the hydrophobic portion 126. In embodiments, the hydrophilic moiety of the hydrophilic portion 124 includes NH₂, COOH, or biotin.

The linker may be provided in any suitable length for separating or spacing the hydrophobic portion 126 at a preselected or threshold distance from the distal surface 106. In embodiments, the linker may be provided as a hydrophilic component or a hydrophilic component that is selected based on chemical properties of the hydrophilic moiety and/or the hydrophobic portion 126. In embodiments, the linker may include polyethylene glycol (PEG).

In embodiments, the amphiphilic polymers 122 and core component 104 are each selected based on their suitability for the immobilization process. In embodiments having the core component 104 including APS, at least one amine or amino group of the APS may covalently bond with an amine group of the hydrophilic moiety of the hydrophilic portion 124 of the amphiphilic polymer 122. In some embodiments, a suitable polar functional group or binding group of the core component 104 may bond, covalently bond, or crosslink to the hydrophilic moiety of the hydrophilic portion 124. Accordingly, the hydrophobic portion 126 of the amphiphilic polymer 122 is spaced from the distal surface 106 of biosensor 100 by a length of the hydrophilic portion 124.

As illustrated in a third phase 140, the hydrophilic portion 124 of the amphiphilic polymer 122 is retained against the distal surface 106 of the biosensor 100, such as based on coupling between the hydrophilic moiety of the amphiphilic polymer 122 and the binding group of the core component 104. The hydrophobic portion 126 is therefore indirectly positioned or immobilized on the distal surface 106 of the core component 104. As such, after introduction of the biosensor 100 into well or a sample fluid containing an analyte 142, the analyte 142 may bind with or be captured by the hydrophobic portion 126 of the amphiphilic polymer 122. The analyte 142 may be a protein analyte, such as an aggregate or mass of a suitably hydrophobic protein. In embodiments, the analyte 142 is an aggregate of an immunoglobulin including IgG. The binding may generally be controlled based on hydrophobic interactions, which, without wishing to be bound by theory, may push, pull, or sequester non-polar components together to reduce interference with hydrogen bonding of polar molecules in the sample fluid. In other words, the hydrophobic portion 126 of the amphiphilic polymer 122 may facilitate capture of the analyte 142 via production of a hydrophobic region that excludes or applies force against polar, hydrophilic compounds.

The disclosed techniques may be particularly efficient for analyzing a presence and/or an amount of protein aggregates in a sample fluid, compared to more costly or intensive analysis via traditional antigen-antibody pairing or ligand-receptor pairing. For example, in certain embodiments, aggregated antibodies may have at least partially unwound antigen-receptor sites, which may reduce a capacity of the antibodies to engage in the traditional antigen-antibody pairing relied upon by traditional analysis techniques.

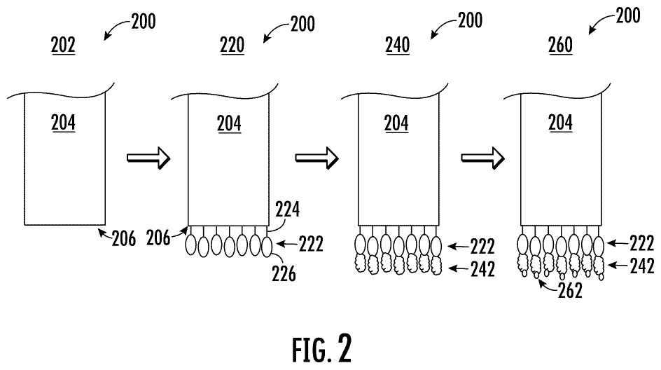

FIG. 2 schematically depicts another embodiment of a process of coating a biosensor, such as a biosensor 200, with amphiphilic polymers for capturing protein aggregates. In the illustrated embodiment, the biosensor 200 is coated with an amphiphilic polymer that acts as an anti-analyte for capturing an analyte, in a manner that is similar to the biosensor 100 of FIG. 1. For example, a first phase 202 of FIG. 2 illustrates the biosensor 200 including a core component 204 having a distal surface 206 that, in a second phase 220, may be coated with amphiphilic polymers 222 each including at least one hydrophilic portion 224 and at least one hydrophobic portion 226. As noted above, the hydrophilic portion 224 may include (i) a hydrophilic moiety coupled to a binding group of the core component 204 and (ii) a linker coupled between the hydrophilic moiety and the hydrophobic portion 226. Additionally, a third phase 240 illustrates capture of an analyte 242, such as a protein aggregate, that is bound with or captured by the hydrophobic portion 226 of the amphiphilic polymer 222.

In a fourth phase 260, the captured analyte 242 of the biosensor 200 is labeled or tagged with a fluorescent dye 262. For example, the biosensor 200 may be introduced to a well or a sample of the fluorescent dye 262, which binds to the captured analyte 242 for facilitating analysis thereof. The fluorescent dye 262 may be selected as any suitable compound having sufficient binding affinity for the captured analyte 242, which includes aggregated protein. For example, in certain embodiments, the fluorescent dye 262 is Congo Red (CR) dye or Thioflavin T (TFT) dye.

Based on the binding interaction between the fluorescent dye 262 and the analyte 242, an efficient quantification of the analyte 242 that was captured via the amphiphilic polymer 222 may be determined via a plate reading device that analyzes a fluorescence intensity, binding rate, and/or response at equilibrium of the captured fluorescent dye 262. As recognized herein, the binding of the fluorescent dye 262 to the distal surface 206 of the biosensor 200 (via the immobilized, amphiphilic polymer 222 and captured analyte 242) facilitates a desirable reduction or even elimination of background fluorescence from unbound fluorescent dyes, such as those found in diagnostic tests performed solely in solution.

FIG. 3 depicts a detailed schematic view of a biosensor 300 for capturing aggregated proteins via an amphiphilic polymer 302. In the illustrated embodiment, the amphiphilic polymer 302 includes a hydrophilic portion 304 and a hydrophobic portion 306. The hydrophilic portion 304 may include (i) at least one hydrophilic moiety 307, such as NH₂, COOH, or biotin, and (ii) a linker 308. As such, the hydrophilic moiety 307 of the hydrophilic portion 304 may be bonded to one or more suitable binding groups 309 of a distal surface 310 or sensing surface of the biosensor 300. The binding group 309 may include a complementary functional group or moiety that covalently bonds with the hydrophilic moiety 307, in certain embodiments. For example, in embodiments in which the distal surface 310 of the biosensor 300 includes APS, an amine group of the APS may be the binding group 309 that covalently bonds or crosslinks with an amine group of the hydrophilic moiety 307. In embodiments, the hydrophobic portion 306 is attached to the linker 308, which is attached to the hydrophilic moiety 307, which is covalently bonded to the binding group 309 of the distal surface 310.

In some embodiments, the linker 308 of the hydrophilic portion 304 includes a single linker arm or an unbranched shape. As such, the hydrophilic portion 304 functions as a linker or spacer for positioning the singular hydrophobic portion 306 at least a threshold distance from the distal surface 310. As discussed below, certain embodiments include hydrophilic portions 304 that are each capable of supporting multiple hydrophobic portions 306. With the embodiments disclosed herein, the hydrophobic portion 306 may therefore capture or bind to an analyte 320 in solution, based on the hydrophobic effect. For example, a hydrophobic region 322 may be formed around the analyte 320 and the hydrophobic portion 306, which excludes polar molecules of an aqueous solution 324 originally containing the analyte 320. Although illustrated around the single amphiphilic polymer 302, it should be understood that the hydrophobic region 322 may extend around any one or more hydrophobic portions 306 and/or bound analytes 320 of one or more amphiphilic polymers 302. That is, these hydrophobic interactions may be occurring across a full area of the biosensor 300 to provide a reliable and efficient mechanism for capture of protein aggregates from solution.

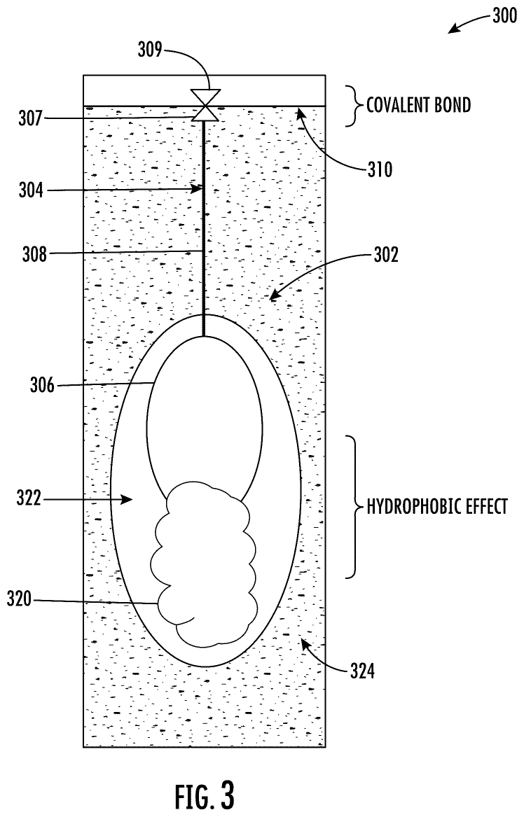

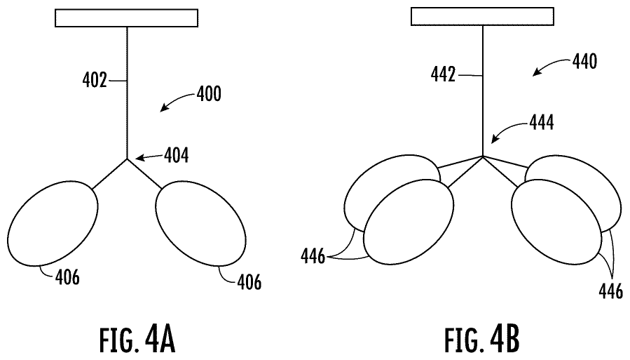

Various properties of the biosensors disclosed herein may be specifically modified for various testing demands. For example, FIG. 4A is a schematic view of an immobilized amphiphilic polymer 400 having a hydrophilic portion 402 with two linker arms 404, thus supporting two hydrophobic portions 406 for analyte detection. FIG. 4B is a schematic view of another immobilized amphiphilic polymer 440 having a hydrophilic portion 442 with four linker arms 444 to support four hydrophobic portions 446. The illustrated arrangement of the linker arms 404, 444 is an example, and it should be understood that other arrangements or configurations may be provided. For example, instead of stemming from a single branch point, other amphiphilic polymers may include linker arms staggered or spaced along a length of the respective hydrophilic portions.

The amphiphilic polymers 400, 440 disclosed herein may have a ratio of hydrophilic portions 402, 442 to hydrophobic portions 406, 446 including 1:1, 1:2, 1:3, 1:4, 1:5, 1:6, 1:7, 1:8, and so on. In some cases, an increased ratio of hydrophobic portions 406, 446 may improve a binding affinity of an analyte, such as a protein aggregate, to an associated biosensor. In embodiments, the increased ratio of hydrophobic portions 406, 446 may increase an upper testing limit of the biosensor by providing additional binding sites between the biosensor and the analyte. The increased ratio of hydrophobic portions 406, 446 may also support or facilitate capture of larger or heavier analytes that may interact with multiple hydrophobic portions simultaneously.

### 2. Analyte Quantitation Apparatus Comprising a Biosensor

An apparatus of the present disclosure, for quantitating an aggregated protein in a sample, includes a biosensor and a signal analyzer. The biosensor includes a core component and a plurality of amphiphilic polymers. Each amphiphilic polymer of the plurality of amphiphilic polymers includes a hydrophilic moiety coupled to a distal surface of the core component and a linker coupled to the hydrophilic moiety. Each amphiphilic polymer includes at least one hydrophobic portion coupled to the linker to facilitate capture of the aggregated protein in the sample via hydrophobic interactions. The aggregated protein may be labeled with a fluorescent dye to facilitate generating of signals indicative of quantity of the aggregated protein captured by the biosensor. The signal analyzer is coupled to the biosensor and is capable of transforming and quantifying the signals generated by labeling of the aggregated protein captured by the biosensor.

In some embodiments, the signal analyzer quantifies the signals generated by labeling of the aggregated protein based on a predetermined calibration curve describing a relationship between aggregated protein concentrations and interaction parameters, such as binding rate, initial slope, and response at equilibrium. In embodiments, the amphiphilic polymer component is immobilized on the distal surface of the core component, and the signal analyzer performs a solid-phase assay. In certain embodiments, the hydrophilic moiety is covalently bonded to the distal surface of the core component. In some embodiments, the at least one hydrophobic portion coupled to the linker includes two or more hydrophobic portions. In certain embodiments, the at least one hydrophobic portion includes DSPE or FMOC.

In more detail, the hydrophobic portion of the amphiphilic polymer is generally non-polar and thus attracts the similarly non-polar protein aggregates in solution for binding or coupling thereto. This binding may generate signals indicative of an amount of the protein aggregate bound to the biosensor. The signal analyzer is capable of quantifying the signals generated by binding of the biosensor to the protein aggregate and/or monitoring and analyzing the binding kinetics over time of the protein aggregate to the biosensor. In embodiments, the signal analyzer quantifies signals by biolayer interferometry (BLI) or using other label-free technology for measuring biomolecular interactions.

In some embodiments, the biosensor may include an optical fiber having a proximal end portion and a distal end portion. The proximal end portion may be configured to receive light from a light source and configured to deliver reflected light to the detector. The optical fiber may be a circular optical fiber having a round cross-section. The distal end portion may be configured to have analytes bind thereto such that light reflected from the distal end portion is phase shifted based on a thickness of analytes bound to the distal end portion. The biosensor of certain embodiments may further include an optical resonator at a distal end portion of the optical fiber, the optical resonator including a first reflective surface and a second reflective surface, the first reflective surface configured to reflect light with a first phase and the second reflective surface configured to reflect light with a second phase which is phase shifted based on a thickness of analytes bound to the optical resonator. The BLI sensor or optical assembly functions as a detector, sensing element, or detector tip to detect analytes attached to an end thereof. The detector senses interference signals produced by interfering light waves reflected from the optical assembly. The detector may be a spectrometer, such as charge-coupled device (CCD), capable of recording the spectrum of the reflected interfering light from the optical assembly.

### 3. Methods for Quantitating a Protein Aggregate

A method is provided by the present disclosure for determining a presence or an amount of protein aggregates in a sample. The method includes contacting the sample with a biosensor. The biosensor includes a core component and a plurality of amphiphilic polymers layered on a distal surface of the core component. Each amphiphilic polymer of the plurality of amphiphilic polymers includes a hydrophilic moiety coupled to the core component, a linker coupled to the core component, and a hydrophobic portion coupled to the linker. The method also includes capturing, via hydrophobic interactions, at least a portion of the protein aggregates in the sample with the hydrophobic portion of the plurality of amphiphilic polymers.

In some embodiments, the method further includes labeling the at least a portion of the protein aggregates with a fluorescent dye. The method includes determining the presence or the amount of the protein aggregates in the sample based on fluorescence intensity associated with the fluorescent dye bound to the protein aggregates. In some embodiments, the method includes determining the presence or the amount of the protein aggregates by measuring the fluorescence intensity and converting the fluorescence intensity to a concentration based on a calibration curve. In embodiments, the fluorescent dye includes Congo Red (CR) dye or Thioflavin T (TFT) dye.

In certain embodiments, the method includes determining the presence or the amount of the protein analyte by measuring interaction parameters based on the binding of protein analyte to the amphiphilic polymers on the biosensor and converting the interaction parameters to a concentration based on a calibration curve. In embodiments, the interaction parameters include a binding rate, an initial slope, and/or a response at equilibrium. In some embodiments, the protein analyte includes aggregated IgG. In some embodiments, the hydrophobic portion of the plurality of amphiphilic polymers comprises DSPE, FMOC, or OPSS. In some embodiments, the hydrophilic moiety of the plurality of amphiphilic polymers is covalently bonded to a binding group of the distal surface of the core component.

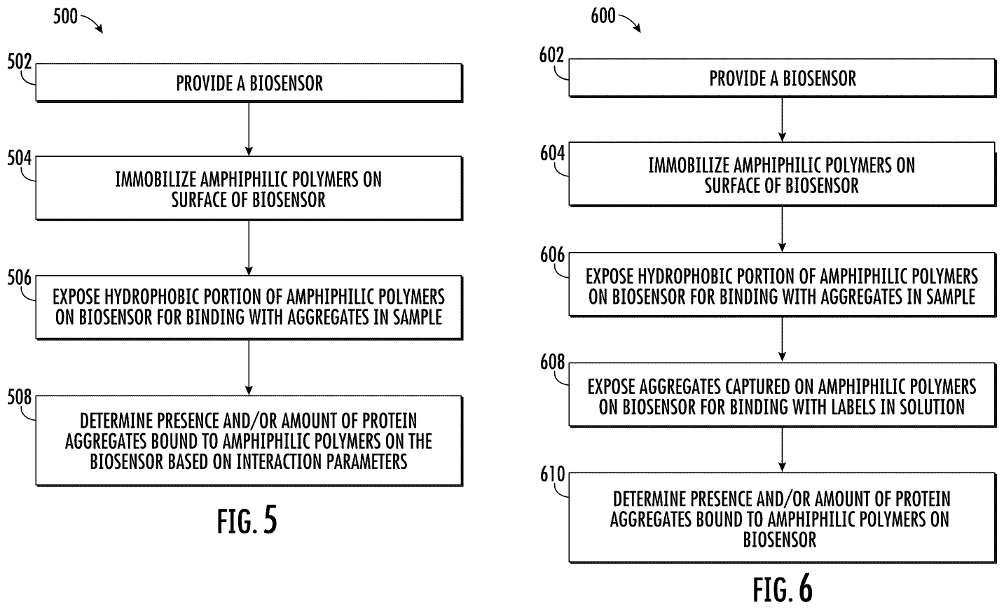

As further illustration, FIG. 5 depicts an example process 500 of detecting the presence or amount of protein aggregates in a sample, according to embodiments of the present disclosure. The process 500 begins, at step 502, by providing a biosensor. At step 504, the process 500 includes immobilizing amphiphilic polymers on a surface of the biosensor. As discussed above, the amphiphilic polymers may generally include at least one hydrophilic moiety to bond with the biosensor, at least one linker coupled to the hydrophilic moiety, and at least one hydrophobic portion coupled to the linker. The process 500 further includes, at step 506, exposing the hydrophobic portion of the amphiphilic polymers on the biosensor for binding with aggregates in a sample. At step 508, the process 500 includes determining a presence and/or an amount of protein aggregates bound to the amphiphilic polymers on the biosensor based on interaction parameters between the biosensor and the protein aggregate, such as binding rate, initial slope, and response at equilibrium. The signals generated by binding or binding kinetics of protein aggregates to the biosensor may be measured by biolayer interferometry (BLI) or other label-free techniques for measuring biomolecular interactions. As such, the method provided herein may be used to detect the presence and/or amount of a protein aggregate in a sample without a detectable label attached to the analyte, either directly or indirectly. The process 500 may generally correspond to the schematic depiction of the biosensor 100 of FIG. 1 having the analyte 142 bound to the hydrophobic portions 126 of the amphiphilic polymers 122, in certain embodiments.

FIG. 6 depicts an example process 600 of detecting the presence or amount of protein aggregates in a sample with a detectable label, according to embodiments of the present disclosure. The process 600 of certain embodiments includes steps 602, 604, and 606 that correspond to steps 502, 504, and 506, which are discussed above and, for brevity, are not repeated here. At step 608, a label, such as a fluorescent dye, is bound to each of the aggregates captured on the amphiphilic polymers. At step 610, the process 600 further includes determining a presence and/or amount of protein aggregates bound to the amphiphilic polymers on the biosensor. This step may be done by measuring the fluorescence intensity associated with the fluorescent dyes bound to the aggregates. The fluorescence intensity may be measured by a plate reader. In some examples, the presence and/or amount of bound protein aggregates is determined based on a binding rate, response at equilibrium, and/or other binding kinetics, in addition or alternative to the fluorescence intensity. The process 600 may generally correspond to the schematic depiction of the biosensor 200 of FIG. 2 having the analyte 242 captured by amphiphilic polymers 222 and labeled with fluorescent dye 262, in some embodiments.

As recognized herein, use of the fluorescent dyes may improve the accuracy, precision, and/or measurement time for analyzing binding characteristics between the analyte and the amphiphilic polymers, compared to embodiments without labels. Also, detecting fluorescence intensity from fluorescent dyes bound to a solid surface, such as a biosensor, improves the accuracy and precision of the results by reducing background signal. For example, the binding of the fluorescent dyes to the biosensor facilitates a desirable reduction or even elimination of background fluorescence from unbound fluorescent dyes, such as those found in diagnostic tests performed with fluorescent dyes remaining in solution.

Another method is provided by the present disclosure for preparing a biosensor for analyzing protein aggregates in a sample. The method includes providing a core component having a distal surface, where the distal surface includes a plurality of binding groups. The method also includes providing a plurality of amphiphilic polymers. Each amphiphilic polymer of the plurality of amphiphilic polymers includes a hydrophilic moiety, a linker coupled to the hydrophilic moiety, and a hydrophobic portion coupled to the linker. Additionally, the method includes exposing the distal surface of the core component to the plurality of amphiphilic polymers, such that the hydrophilic moiety of the plurality of amphiphilic polymers binds to the plurality of binding groups of the distal surface and the hydrophobic portion of the plurality of amphiphilic polymers is immobilized to facilitate analysis of protein aggregates.

In some embodiments, the hydrophilic moiety of the plurality of amphiphilic polymers or the binding group of the distal surface includes an amine group. In some embodiments, the hydrophobic portion of the plurality of amphiphilic polymers includes DSPE, FMOC, or OPSS. In some embodiments, the method includes preparing the plurality of amphiphilic polymers by binding the hydrophilic moiety to a first end of the linker and binding the hydrophobic portion to a second end of the linker. In embodiments, the method includes capturing, via hydrophobic interactions, at least a portion of protein aggregates in a sample with the hydrophobic portion of the plurality of amphiphilic polymers, labeling the at least a portion of the protein aggregates with a fluorescent dye, and determining a presence or an amount of the protein aggregates in the sample based on signals emitted from the fluorescent dye.

In other embodiments, a presence or amount of protein aggregates in a sample may be detected using a reagent in the sample that is configured to react with an analyte on a biosensor. Reagents configured to react with analytes on a biosensor may generate an optical signal upon undergoing such a reaction. For example, a sample may comprise a reagent configured to undergo a reaction with an analyte immobilized on a biosensor that generates an analyte that absorbs light, transmits lights, reflects light, fluoresces light, undergoes scattering (e.g., Raman scattering), is polarized, and/or undergoes luminescence.

Non-limiting examples of reagents that may be configured to generate an optical signal include enzyme substrates (e.g., for enzyme-linked antibodies). Some methods comprise reacting an enzyme substrate with an enzyme (e.g., an enzyme-linked antibody). Such a reaction may result in the generation of products of an enzymatic reaction, one or more of which may be capable of and/or configured to generate an optical signal related to or indicative of immobilization of the analyte on the biosensor.

In other embodiments, a presence or amount of protein aggregates in a sample may be detected from measurements of two optical signals in one instrument. The first optical signal may be generated from BLI, as described above, and the second optical signal may be generated from an analyte that absorbs light, transmits lights, reflects light, fluoresces light, undergoes scattering (e.g., Raman scattering), is polarized, and/or undergoes luminescence in response to reaction with a reagent in the sample.

In some embodiments, the instrument has first and second light sources and first and second optical detectors, respectively. The first optical detector is configured to detect a first type of optical signal and the second optical detector is configured to detect a second type of optical signal.

### EXAMPLES

### Example 1: Evaluating Fluorescence Intensity and Generating a Calibration Curve for a Biosensor Having a First Amphiphilic Polymer

This example relates to evaluating fluorescence intensity of various standard solutions to generate a calibration curve for a biosensor suitable for characterizing protein aggregates. An amphiphilic polymer was immobilized onto a distal surface of a biosensor containing APS. After the immobilization process, hydrophilic moieties of the amphiphilic polymer are bound to the APS of the biosensor, and linkers are coupled between the hydrophilic moieties and hydrophobic portions or heads of the amphiphilic polymer. As such, the hydrophobic portions are positioned for subsequent binding with analytes, such as protein aggregates. The biosensor was dipped into a well plate (e.g., 96 well plate or 384 well plate) having a respective standard solution for 15 minutes. Subsequently, the biosensor was further dipped into a well with fluorescent dye where the captured analytes on the biosensor were tagged with a suitable fluorescent dye. In this example, the hydrophobic portions of the amphiphilic polymer included DSPE. A sample of human IgG at 4 mg/mL was incubated in a low pH buffer (0.1M Glycine-HCl, pH 3.0) at 50° C. overnight to aggregate. Then, a 100% IgG aggregates standard having 1000 μg/mL in PBS was acquired and diluted with native human IgG monomers to various percentages of aggregates. In particular, standard samples having 90%, 70%, 50%, 30%, 20%, 10%, and 5% aggregates were prepared. Each standard was evaluated via a prepared biosensor with a plate reader.

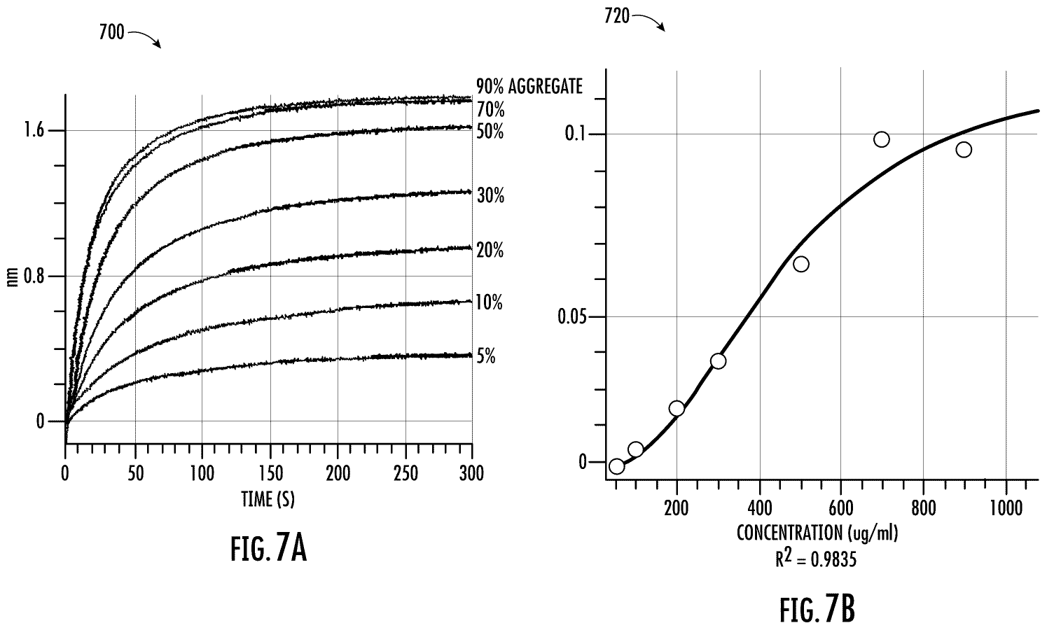

For example, FIG. 7A is a graphical representation of multiple binding curves 700, illustrating relationships of a change in wavelength (nm shift) versus time (in seconds). Each curve of FIG. 7A represents the measured nm shift over time for a specific aggregate to monomer percentage, denoted on the right-hand side of the respective binding curve. The fluorescence intensity measurements indicate that higher percentages of aggregates experience a greater nm shift during the loading time frame, such as based on a higher occupancy of the aggregates on the hydrophobic portions of the biosensor.

Moreover, FIG. 7B is a graphical representation of an embodiment of a calibration curve 720, which depicts a relationship of binding rate versus aggregate concentration. The calibration curve 720 is generated based on the binding curves 700, such as by plotting various data points based on binding kinetics for aggregate concentrations and the associated nm shift. The calibration curve 720 facilitates determination of aggregated protein concentrations in subsequently tested samples, as described herein. Accordingly, the present techniques facilitate efficient evaluation and characterization of protein aggregates based on predetermined calibration curves.

### Example 2: Evaluating Fluorescence Intensity and Generating a Calibration Curve for a Biosensor Having a Second Amphiphilic Polymer

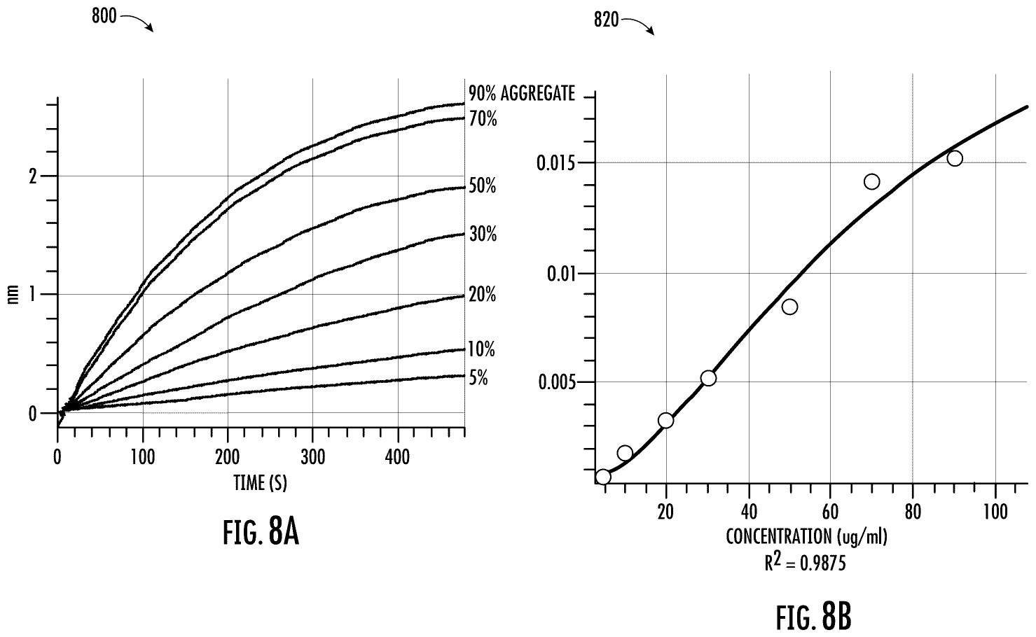

This example relates to evaluating fluorescence intensity of various standard solutions to generate a calibration curve for another biosensor suitable for characterizing protein aggregates. The testing procedure was similar to the above-provided procedure for Example 1, with two main differences. First, the hydrophobic portions of the amphiphilic polymer immobilized on the biosensor included FMOC instead of DSPE. Second, the 100% IgG aggregates standard for dilution had 100 ug/mL in PBS instead of 1000 ug/mL. The standards were again prepared at concentrations of 90%, 70%, 50%, 30%, 20%, 10%, and 5% aggregates, and evaluated via the present techniques. For example, FIG. 8A is a graphical representation of multiple binding curves 800, illustrating relationships of nm shift versus time (in seconds) for the biosensor of Example 2. Additionally, FIG. 8B is a graphical representation of an embodiment of a calibration curve 820, which depicts a relationship of binding rate versus aggregate concentration for the biosensor of Example 2. The calibration curve 820 thus enables evaluation of the characteristics and concentrations of future samples of aggregated protein via biosensors having the amphiphilic polymer coated thereon.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as examples of particular embodiments. Those skilled in the art will envision other modifications within the scope of the claims appended hereto.

What is claimed:

1. A biosensor for detecting a presence or amount of protein aggregates in a sample, the biosensor comprising:
a core component comprising a distal surface; and
a plurality of amphiphilic polymers, wherein each amphiphilic polymer of the plurality of amphiphilic polymers comprises:
a linker having a first end and a second end;
a hydrophilic moiety coupled between the first end of the linker and the distal surface of the core component; and
a hydrophobic portion coupled to the second end of the linker.

2. The biosensor of claim 1, wherein the hydrophobic portion of each amphiphilic polymer facilitates capture of the protein aggregates in the sample via hydrophobic interactions and facilitates generating of signals indicative of an amount of the protein aggregates captured by the biosensor.

3. The biosensor of claim 2, wherein the hydrophobic portion of each amphiphilic polymer facilitates the capture of the protein aggregates via production of a hydrophobic region for sequestering non-polar components therein.

4. The biosensor of claim 2, wherein a fluorescent dye is bound to the captured protein aggregates of each amphiphilic polymer.

5. The biosensor of claim 1, wherein the linker comprises a single linker arm.

6. The biosensor of claim 1, wherein the linker comprises a plurality of linker arms, and wherein each linker arm of the plurality of linker arms is coupled to a respective hydrophobic portion of a plurality of hydrophobic portions.

7. The biosensor of claim 1, wherein the hydrophobic portion comprises a phospholipid or an amine protecting group.

8. The biosensor of claim 1, wherein the hydrophobic portion comprises 1,2-distearoyl-sn-glycero-3-phosphorylethanolamine (DSPE).

9. The biosensor of claim 1, wherein the hydrophobic portion comprises fluorenylmethoxycarbonyl (FMOC).

10. The biosensor of claim 1, wherein the distal surface of the core component comprises aminopropylsilane (APS).

11. The biosensor of claim 1, wherein the protein aggregates comprise aggregates of immunoglobulin G (IgG).

12. The biosensor of claim 1, wherein the linker comprises polyethylene glycol (PEG).

13. A method for determining a presence or an amount of protein aggregates in a sample, the method comprising:
contacting the sample with a biosensor, the biosensor comprising a core component and a plurality of amphiphilic polymers layered on a distal surface of the core component, each amphiphilic polymer of the plurality of amphiphilic polymers comprising a hydrophilic moiety coupled to the core component, a linker coupled to the hydrophilic moiety, and a hydrophobic portion coupled to the linker;
capturing, via hydrophobic interactions, at least a portion of the protein aggregates in the sample with the hydrophobic portion of the plurality of amphiphilic polymers;
labeling the at least a portion of the protein aggregates with a fluorescent dye; and
determining the presence or the amount of the protein aggregates in the sample based on fluorescence intensity associated with the fluorescent dye bound to the protein aggregates.

14. The method of claim 13, wherein determining the presence or the amount of the protein aggregates comprises:
measuring the fluorescence intensity; and
converting the fluorescence intensity to a concentration based on a calibration curve.

15. The method of claim 13, wherein determining the presence or the amount of the protein aggregates comprises measuring a binding rate, initial slope, response at equilibrium, or combination thereof based on the binding of protein aggregates to the hydrophobic portion of the plurality of amphiphilic polymers.

16. The method of claim 13, wherein the protein aggregates comprise aggregated IgG.

17. The method of claim 13, wherein the fluorescent dye comprises Congo Red (CR) dye or Thioflavin T (TFT) dye.

18. The method of claim 13, wherein the hydrophobic portion of the plurality of amphiphilic polymers comprises DSPE, FMOC, or ortho-pyridyl disulfide (OPSS).

19. The method of claim 13, wherein the hydrophilic moiety of the plurality of amphiphilic polymers is covalently bonded to a binding group of the distal surface of the core component.

20. A method for preparing a biosensor for analyzing protein aggregates in a sample, the method comprising:
providing a core component having a distal surface, wherein the distal surface comprises a plurality of binding groups;
providing a plurality of amphiphilic polymers, each amphiphilic polymer of the plurality of amphiphilic polymers comprising a hydrophilic moiety, a linker coupled to the hydrophilic moiety, and a hydrophobic portion coupled to the linker; and
exposing the distal surface of the core component to the plurality of amphiphilic polymers, such that the hydrophilic moiety of the plurality of amphiphilic polymers binds to the plurality of binding groups of the distal surface and the hydrophobic portion of the plurality of amphiphilic polymers is immobilized to facilitate analysis of protein aggregates.

21. The method of claim 20, wherein the hydrophilic moiety of the plurality of amphiphilic polymers or the binding group of the distal surface comprises an amine group.

22. The method of claim 20, wherein the hydrophobic portion of the plurality of amphiphilic polymers comprises DSPE, FMOC, or OPSS.

23. The method of claim 20, comprising preparing the plurality of amphiphilic polymers by binding the hydrophilic moiety to a first end of the linker and binding the hydrophobic portion to a second end of the linker.

24. The method of claim 20, comprising:
capturing, via hydrophobic interactions, at least a portion of protein aggregates in a sample with the hydrophobic portion of the plurality of amphiphilic polymers;
labeling the at least a portion of the protein aggregates with a fluorescent dye; and
determining a presence or an amount of the protein aggregates in the sample based on signals emitted from the fluorescent dye.
